(19) United States
(12) Patent Application Publication
(10) Pub. No.: US 2026/0241203 A1
(43) Pub. Date: Aug. 20, 2026

(54) METHOD AND APPARATUS FOR GENERATING AND REVIEWING AN ENERGY TREATMENT PLAN

(71) Applicant: Siemens Healthineers International AG, Steinhausen (CH)

(72) Inventors: Kellee Donnelly, Helsinki (FI); Martin Sabel, Hagendorn (CH); Elena Czeizler, Helsinki (FI)

(21) Appl. No.: 19/058,256

(22) Filed: Feb. 20, 2025

(51) Int. Cl. A61N 5/10

(52) U.S. Cl. CPC A61N 5/1031; A61N 2005/1041; A61N 2005/1074; A61N 2005/1087

(57) ABSTRACT

A control circuit that is operably coupled to a user interface accesses user-entered energy treatment plan-formation information, automatically generates an energy treatment plan for the particular patient using the particular energy treatment apparatus as a function of the user-entered energy treatment plan-formation information to provide an automatically-generated energy treatment plan, and provides a plurality of discrete user-review opportunities for the user to evaluate the automatically-generated energy treatment plan, each of which plurality of discrete user-review opportunities does not include an opportunity for the user to select revisiting the user-entered energy treatment plan-formation information. Subsequent to providing the plurality of discrete user-review opportunities, the control circuit provides a discrete user opportunity that includes an opportunity for the user to select revisiting the user-entered energy treatment plan-formation information and thereby begin again development of the energy treatment plan.

### TECHNICAL FIELD

These teachings relate generally to treating a patient's planning target volume with energy pursuant to an energy-based treatment plan and more particularly to generating and reviewing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

An energy treatment plan typically comprises specified values for each of a variety of treatment-apparatus parameters during each of a plurality of sequential fields. Treatment plans for energy treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

Varying degrees of automation are sometimes applied in the context of generating an energy treatment plan. Automation can offer, at least in some application settings, more rapid generation of a viable plan as compared to a more human-based planning workflow.

### BRIEF DESCRIPTION OF DRAWINGS

The above needs are at least partially met through provision of the method and apparatus for generating and reviewing an energy treatment plan described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a workflow diagram as configured in accordance with a prior art approach;

FIG. 4 comprises a workflow diagram as configured in accordance with various embodiments of these teachings;

FIG. 5 comprises a screenshot as configured in accordance with various embodiments of these teachings;

FIG. 6 comprises a screenshot as configured in accordance with various embodiments of these teachings;

FIG. 7 comprises a screenshot as configured in accordance with various embodiments of these teachings;

FIG. 8 comprises a screenshot as configured in accordance with various embodiments of these teachings; and

FIG. 9 comprises a screenshot as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

The applicant has determined that user trust in automated treatment planning workflows can sometimes be marginal, leading to low acceptance of automatically generated energy treatment plan results. Instead, users may prefer to create plans using more manual approaches, even when it takes longer to complete the task, and even when the plan quality is ostensibly the same or only marginally different than a counterpart automated plan.

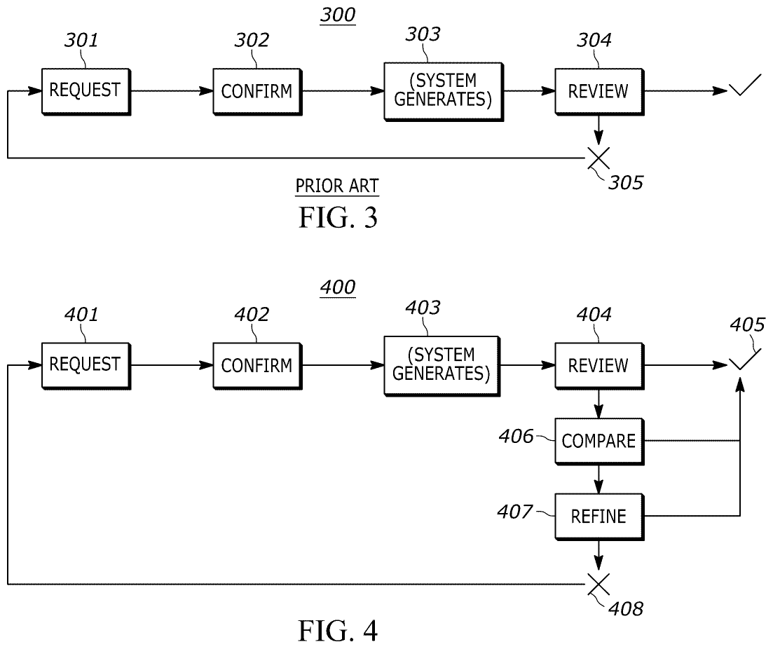

It may be helpful to first describe an example of a prior art automated energy treatment plan workflow. FIG. 3 presents an illustrative example of such a workflow 300.

This workflow 300 begins with the user's request 301 and confirmation 302 of the details of the request 301. Block 303 illustrates automated plan generation followed by user review 304 of the generated energy treatment plan. Should the user find the plan deficient or wanting in any regard, the user rejects the plan 305 and the workflow begins anew at the beginning. Such a workflow 300 allows for user interaction at the start of the workflow 300 during the request 301 and confirm 302 stages, but these opportunities do not necessarily always ensure that user changes (such as, for example, changes to the request regarding added clinical goals, changed clinical goal(s) priority, and/or potentially adding specific optimization structures to control areas of dosing in three-dimensional space) to a given automatically-generated plan workflow will have the desired impact on plan quality. Such an approach can lead to a user feeling detached and substantively left out of the process and with no effective way to communicate their desired result.

Generally speaking, these various embodiments seek to address such considerations. These various embodiments can provide for a control circuit that is operably coupled to a user interface and that is configured to access user-entered energy treatment plan-formation information and then automatically generate an energy treatment plan for a particular patient using a particular energy treatment apparatus as a function of the user-entered energy treatment plan-formation information to provide an automatically-generated energy treatment plan. The control circuit can then provide a plurality of discrete user-review opportunities for the user to evaluate the automatically-generated energy treatment plan, each of which plurality of discrete user-review opportunities does not include an opportunity for the user to select revisiting the user-entered energy treatment plan-formation information. In addition, the control circuit can, subsequent to providing the aforementioned plurality of discrete user-review opportunities, provide a discrete user opportunity that includes an opportunity for the user to select revisiting the user-entered energy treatment plan-formation information and thereby begin again development of the energy treatment plan. That revisiting can comprise starting genuinely anew, or, if desired, can comprise leveraging one or more already achieved planning results.

By one approach, the user-entered energy treatment plan-formation information includes at least one of patient target volume information, prescribed dose information, and dosing clinical goals.

By one approach, automatically generating the energy treatment plan for the particular patient using the particular energy treatment apparatus as a function of the user-entered energy treatment plan-formation information can comprise optimization such that the automatically-generated energy treatment plan comprises an optimized energy treatment plan.

By one approach, one of the aforementioned plurality of discrete user-review opportunities for the user can comprise an automatically-generated energy treatment plan quality review. That review, in turn, may comprise a presentation, via the aforementioned user interface, of one or more of dose-based patient imagery, clinical goals compliance, and at least one dose-volume histogram. By one approach, that presentation may comprise a read-only presentation that includes a user-opportunity to approve the automatically-generated energy treatment plan.

By one approach, one of the aforementioned plurality of discrete user-review opportunities for the user can comprise a historical information comparison opportunity such as, for example, a comparative presentation of dose information for at least one historical energy treatment session. By one approach, if desired, that historical energy treatment session dose information can comprise at least one of dose volume histogram estimates, three-dimensional dose predictions, and/or clinical goal statistics.

And by another approach, in lieu of the foregoing or in combination therewith, one of the plurality of discrete user-review opportunities for the user may comprise an opportunity for the user to modify at least one automatically-generated energy treatment plan result.

So configured, these teachings help build and maintain user trust in automated treatment planning workflows by offering a workflow that allows (and even encourages) intentional user interaction after the user has seen the automated plan result. In particular, a user can compare and/or refine the result. These teachings effectively offer multiple paths to an accepted result, without necessitating going backwards to re-define an initial request.

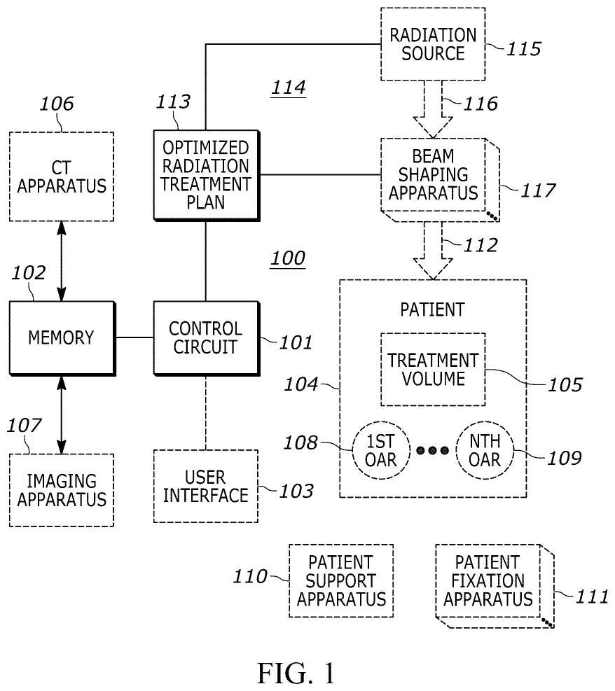

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware apparatus (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware apparatus (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated apparatus or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101). As with the control circuit 101, the memory 102 may comprise a singular structure or may comprise a plurality of memory apparatuses that collectively comprise the “memory” of this apparatus 100.

In addition to information such as optimization information for a particular patient and information regarding a particular radiation treatment apparatus as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless apparatuses, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-apparatus parameters during each of a plurality of sequential exposure fields. In this case the energy-based treatment plan is generated through an optimization process, examples of which are provided further herein.

By one approach the control circuit 101 can operably couple to an energy-based treatment apparatus 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment apparatus/apparatuses. In a typical application setting the energy-based treatment apparatus 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment apparatus 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

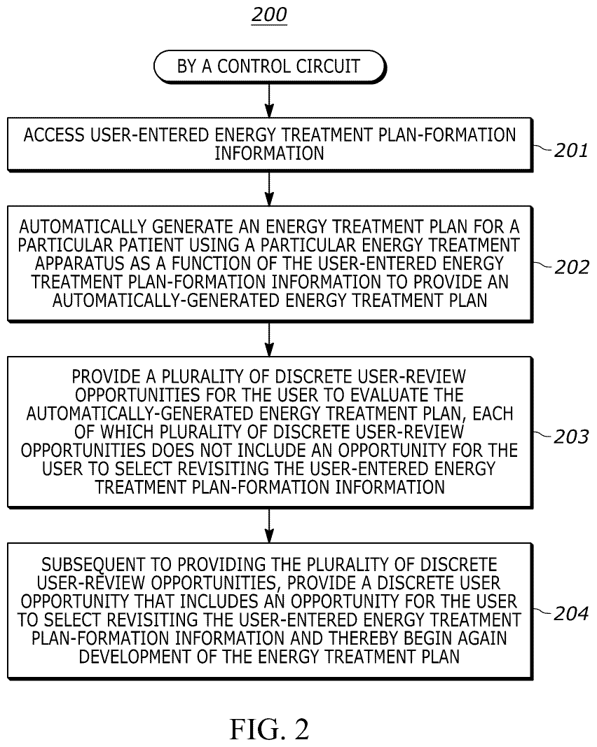

Referring now to FIG. 2, a process 200 that can be carried out, for example, in conjunction with the above-described application setting (and more particularly via the aforementioned control circuit 101) will be described. Generally speaking, this process 200 serves to facilitate generating and reviewing an optimized energy treatment plan (which, for the sake of an illustrative example, is presumed herein to be a radiation treatment plan 113) to thereby facilitate treating a particular patient with therapeutic radiation using a particular radiation treatment apparatus per that optimized radiation treatment plan 113.

At block 201, this process 200 provides for accessing user-entered energy treatment plan-formation information. Examples in these regards include, but are not limited to, one or more of patient target volume information, prescribed dose information, and dosing clinical goals.

At block 202, the control circuit 101 automatically generates an energy treatment plan for a particular patient using a particular energy treatment apparatus as a function of the user-entered energy treatment plan-formation information to provide an automatically-generated energy treatment plan such as, an optimized energy treatment plan. Various approaches are known in the art to optimize an energy treatment plan. As the present teachings are not overly sensitive to any particular selection in these regards, further elaboration will not be provided here regarding optimization for the sake of brevity.

At block 203, the control circuit 101 then provides a plurality of discreet user-review opportunities for the user to evaluate the automatically-generated energy treatment plan. Each of these plurality of discrete user review opportunities does not include an opportunity for the user to select revisiting the user-entered energy treatment plan-formation information. These teachings will accommodate any of a variety of different review opportunities.

By one approach, at least one of the discreet user-review opportunities can comprise an automatically-generated energy treatment plan quality review. Such a review may comprise, for example, a presentation (via the aforementioned user interface 103) of one, some, or all of dose-based patient imagery, clinical goals compliance, and at least one dose-volume histogram. Such a presentation may comprise, for example, a read-only presentation that includes a user opportunity to approve the automatically-generated energy treatment plan.

By another approach, in lieu of the foregoing or in combination therewith, at least one of the discrete user-review opportunities can comprise a historical information comparison opportunity. The latter may comprise, for example, dose information for at least one historical energy treatment session and may comprise at least one of dose volume histogram estimates, three-dimensional dose predictions, and/or clinical goal statistics. Generally speaking, this approach can comprise a review of historical patient data across many patients having a similar disease and/or treatment including historical plans as well as other types of supporting information (including information from the plan generation process such as optimization costs per goal).

By yet another approach, and again in lieu of the foregoing or in combination therewith, at least one of the aforementioned discrete user-review opportunities for the user can comprise an opportunity for the user to modify at least one automatically-generated energy treatment plan result. This can comprise, for example, modification of one or more features to thereby hopefully refine and improve the resultant plan without starting over from the beginning.

At 204, the control circuit 101, subsequent to providing the aforementioned plurality of discrete user-review opportunities, can then provide a discrete user opportunity that includes an opportunity for the user to select revisiting the user-entered energy treatment plan-formation information and thereby begin development of the energy treatment plan.

Further details that comport with these teachings will now be presented. It will be understood that the specific details of these examples are intended to serve an illustrative purpose and are not intended to suggest any particular limitations with respect to these teachings.

Viewed one way, and by one approach, these teachings offer a workflow having additional steps (as compared to the above-described prior art workflow) that enable additional paths for where a user can accept an automatically generated plan. These additional opportunities can, for example, facilitate user decision making (for example, by introducing additional data) and/or allow the user to interact with the automatically generated plan via, for example, dose editing and/or alternative plan selection). That said, the workflow will still permit a user to decide to start again by re-defining their request (that is, for example, their initial input criteria, such as clinical goals, dose prescription(s), and so forth) if needed for the patient case.

FIG. 4 presents an illustrative workflow 400 that comports with the foregoing teachings.

The request at block 401 is where the user defines the essential inputs for treatment planning. Examples in these regards include, but are not limited to, patient anatomy and density information (input, at least in part and by one approach, as one or more computed tomography image and structure contours), target (tumor) size and location within the patient (input, for example, as one or more structure contours), prescribed dose and fractionation scheme information, expected target coverage (included, for example, within clinical goals), and/or prioritized dose constraints that pertain to healthy tissue and organs (again included, for example, within clinical goals).

Confirmation at block 402 can comprise, for example, the user reviewing the aforementioned request information and noting any detected conflicts that may be corrected before continuing. This activity may also include, for example, considering any facilitating information that may help with defining their request (such as, for example, drafted dose information, dose predictions, and/or specifically noted or otherwise presented potential conflicts).

At block 403 the system automatically generates an optimized energy treatment plan. There are numerous prior art approaches that may be used to carry out this step of the workflow 400.

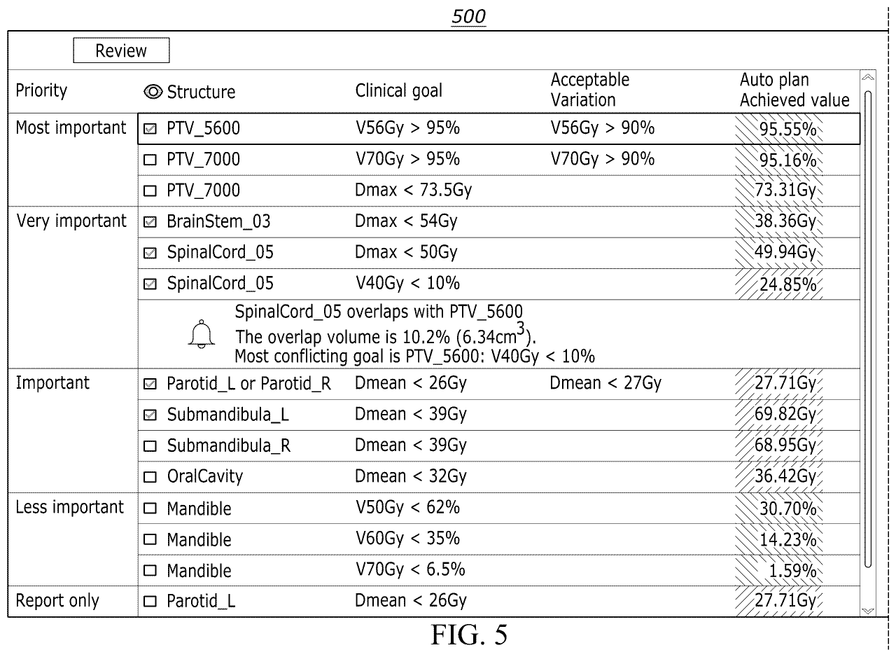

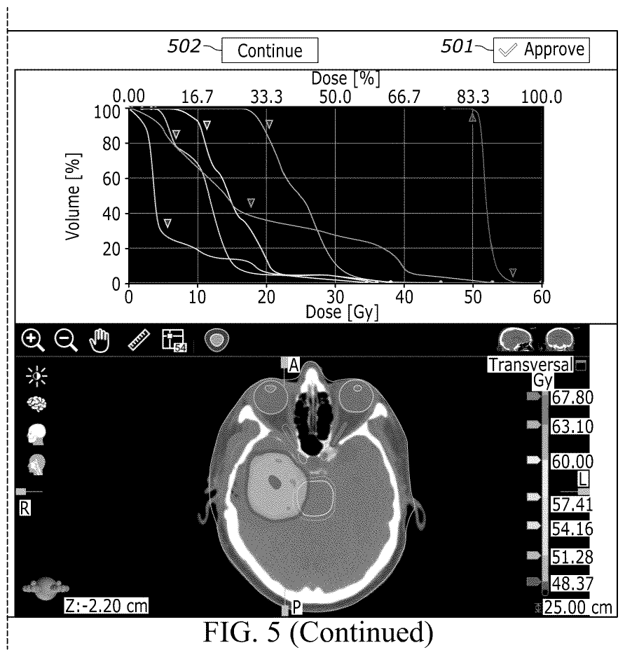

At block 404 the workflow 400 provides a review opportunity. At this review opportunity, the automated planning result can be displayed in a read-only format for the user to review the quality of the plan in terms of the patient case. This review opportunity can employ, as desired, any of a variety of existing tools for viewing dose over computed tomography Images, clinical goals, dose-volume histograms, and so forth. FIG. 5 presents a screenshot 500 of an illustrative example of a review opportunity. The user can accept the plan result (as denoted by reference numeral 405 in FIG. 4) by clicking an “approve” button 501 (and thereby conclude the planning workflow 400) or can choose to continue with the workflow 400 (for example, by clicking a “continue” button 502).

If the plan is not accepted at block 404, the workflow 400 in this example next provides a comparison opportunity at block 406. To reach this point in the workflow 400, the user has reviewed at some level the automated plan result, and is skeptical of the quality of the result. This comparison step allows the user to compare the optimized energy treatment plan with facilitating information. Examples of useful facilitating information include, but are not limited tom, dose information from historical patient cases (including such things as dose volume histogram estimates, three-dimensional dose predictions, clinical goal statistics, and so forth).

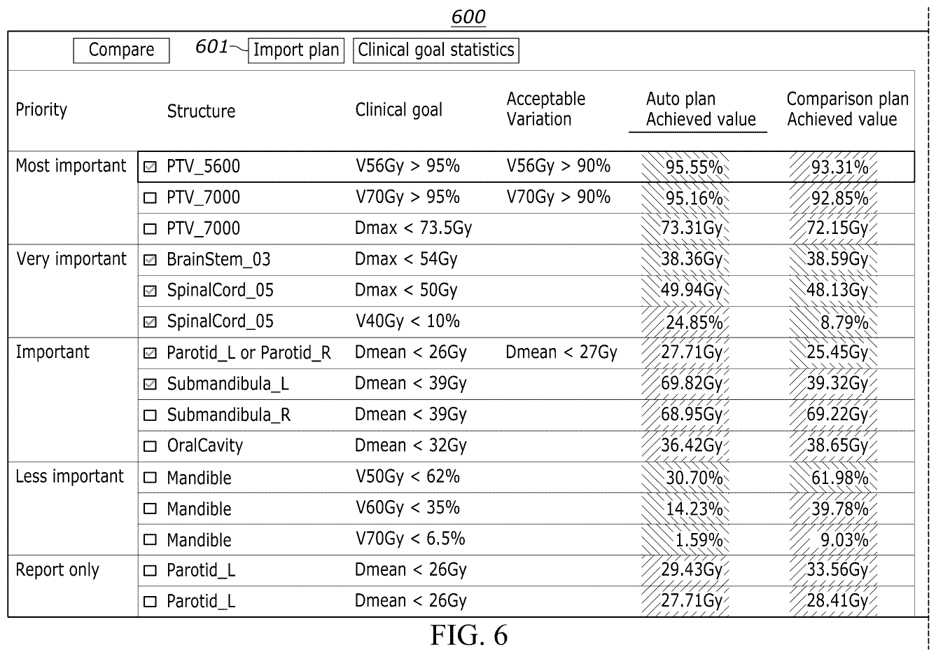

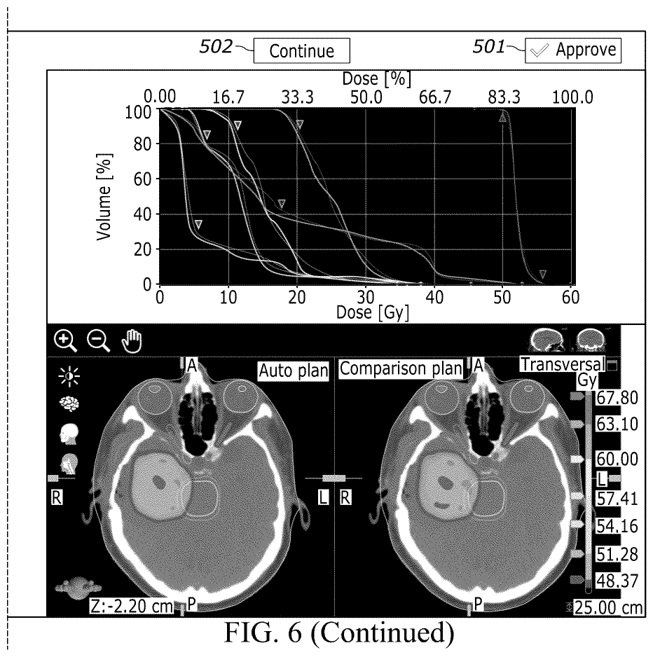

FIG. 6 presents an illustrative screenshot 600 of a comparison opportunity that corresponds to the user clicking on an “import plan” button 601. As before, the user can now choose to accept the plan result for treatment and thus end the planning workflow 400, or not.

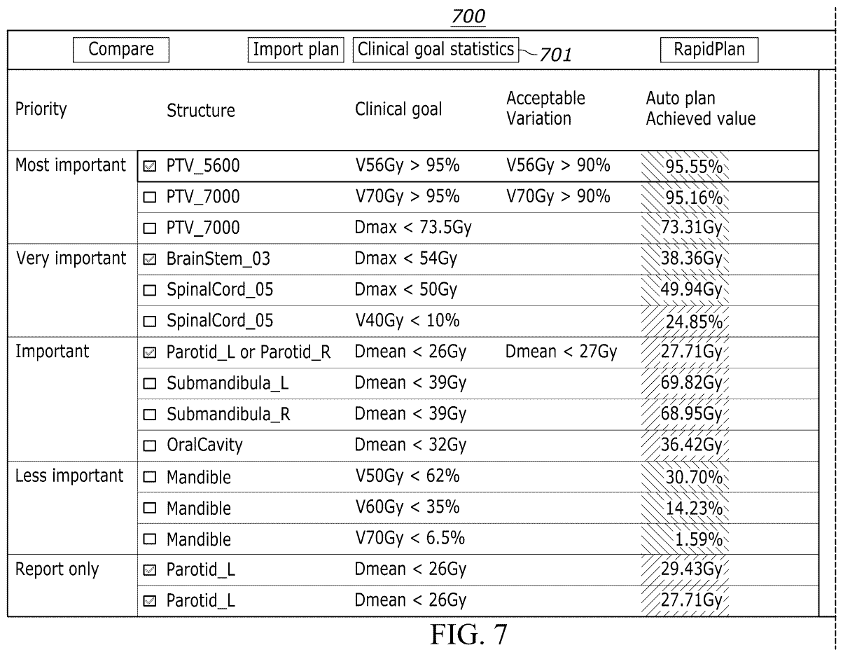

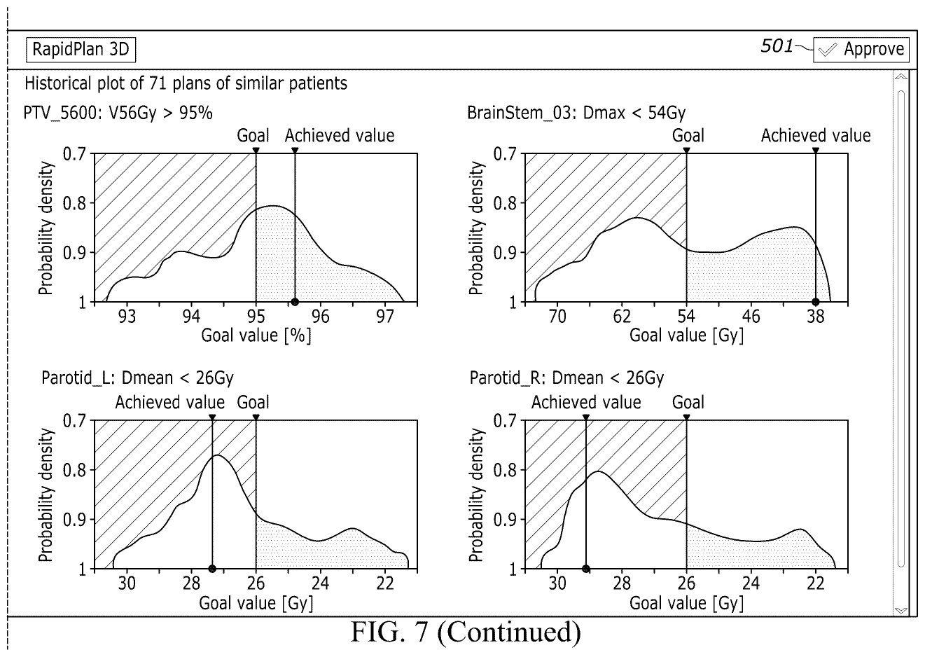

FIG. 7 presents an illustrative screenshot 700 of a comparison opportunity that corresponds to the user clicking on a “clinical goal statistics” button 701. And again, the user can now choose to accept the plan result for treatment and thus end the planning workflow 400, or not.

If after reviewing the optimized plan, and comparing that plan to facilitating information, if the user remains unsure the workflow 400 provides a refinement step at block 407. “Refinement” can refer to changes to the plan that do not amount to beginning anew. Refinement changes can include, for example, such as things as modifying a three-dimensional dose (for example, by decreasing the size of a hot spot, where, for example, an organ-at-risk may be receiving an undue amount of energy), re-prioritizing goals, Pareto front exploration, and/or selecting an alternate result with a different organ-sparing approach (using, for example, a pre-calculated plan alternative). Again, the user can accept the refined plan result for treatment (and end the planning workflow 400), or not. By one approach, the user can be provided with the opportunity to choose between the initial plan result and one or more of their refined results.

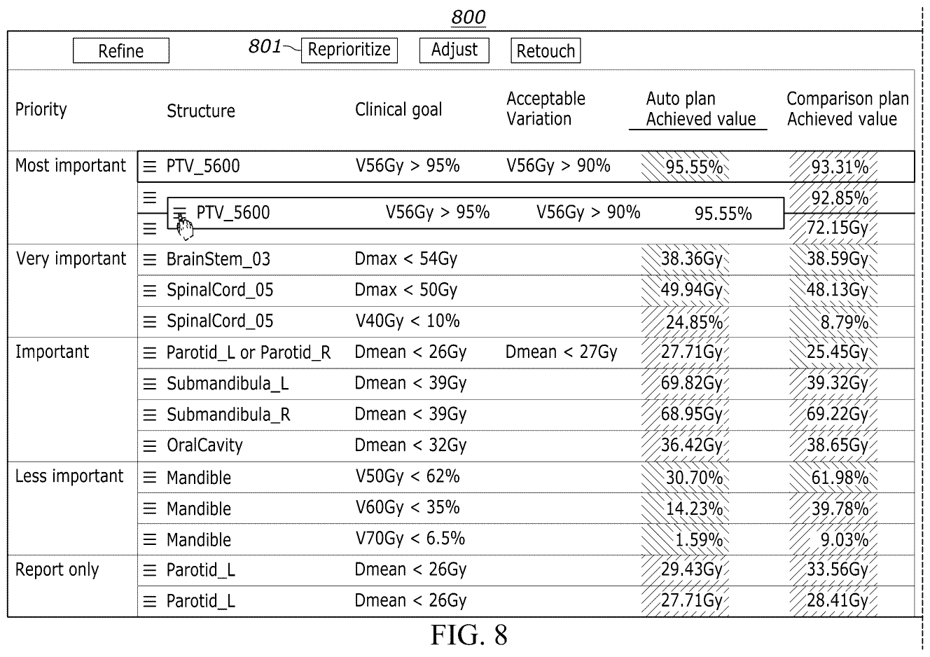

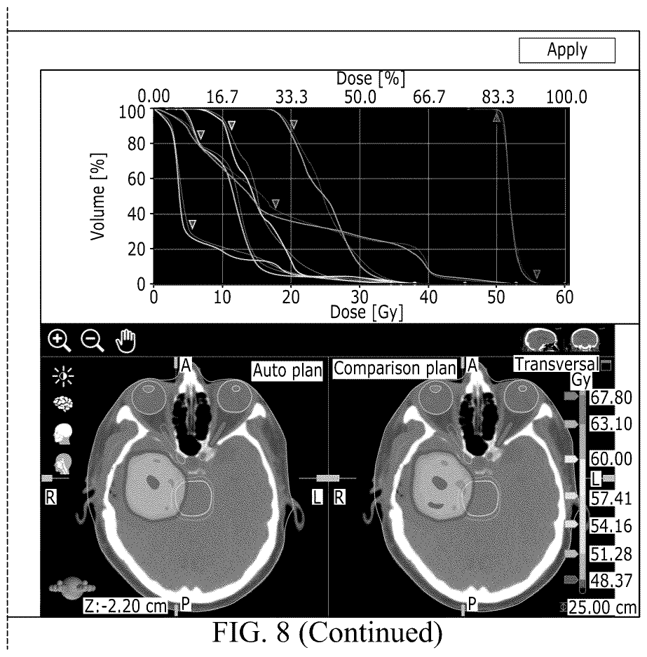

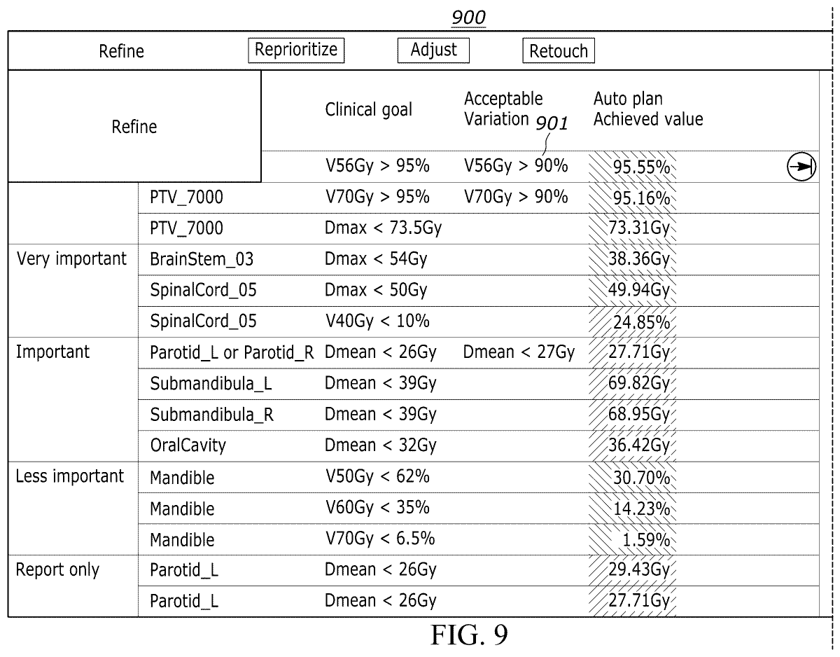

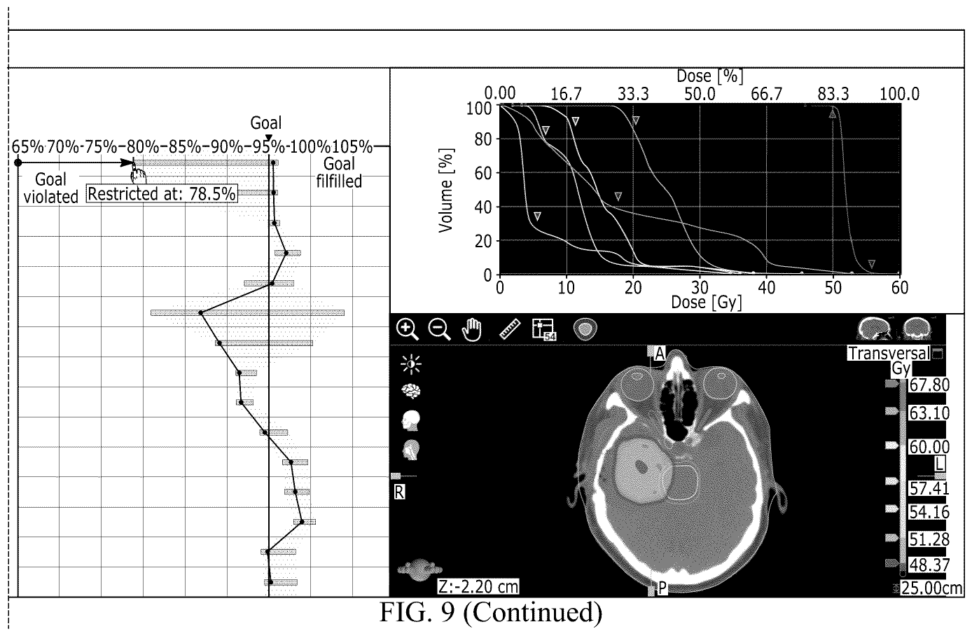

FIG. 8 presents an illustrative screenshot 800 of a refinement opportunity that corresponds to the user having selected a “reprioritize” option 801. And FIG. 9 presents an illustrative screenshot 900 of a refinement opportunity that corresponds to the user having selected an “adjust” option 901.

Should none of the above yield a satisfactory result, as represented by reference numeral 408 in FIG. 4 the workflow 400 returns the user to the starting point, and the process can begin anew. At this point, some or all of the initial request information can be different than what the user originally presented.

Further aspects of these teachings are provided by the subject matter of the following clauses (where it will be understood that any of these clauses can be combined with any one of more of the other clauses as appropriate).

Clause 1. A method for generating an energy treatment plan for a particular patient using a particular energy treatment apparatus, the method comprising: by a control circuit that is operably coupled to a user interface: accessing user-entered energy treatment plan-formation information; automatically generating an energy treatment plan for the particular patient using the particular energy treatment apparatus as a function of the user-entered energy treatment plan-formation information to provide an automatically-generated energy treatment plan; providing a plurality of discrete user-review opportunities for the user to evaluate the automatically-generated energy treatment plan, each of which plurality of discrete user-review opportunities does not include an opportunity for the user to select revisiting the user-entered energy treatment plan-formation information; subsequent to providing the plurality of discrete user-review opportunities, providing a discrete user opportunity that includes an opportunity for the user to select revisiting the user-entered energy treatment plan-formation information and thereby begin again development of the energy treatment plan.

Clause 2. The method of clause 1 wherein the user-entered energy treatment plan-formation information includes at least one of patient target volume information, prescribed dose information, and dosing clinical goals.

Clause 3. The method of either of clause 1 through 2 wherein automatically generating the energy treatment plan for the particular patient using the particular energy treatment apparatus as a function of the user-entered energy treatment plan-formation information comprises optimization such that the automatically-generated energy treatment plan comprises an optimized energy treatment plan.

Clause 4. The method of any of clause 1 through 3 wherein one of the plurality of discrete user-review opportunities for the user comprises an automatically-generated energy treatment plan quality review.

Clause 5. The method of clause 4 wherein the automatically-generated energy treatment plan quality review comprises a presentation, via the user interface, of: dose-based patient imagery; clinical goals compliance; at least one dose-volume histogram.

Clause 6. The method of clause 5 wherein the presentation comprises a read-only presentation that includes a user-opportunity to approve the automatically-generated energy treatment plan.

Clause 7. The method of any of clause 1 through 6 wherein one of the plurality of discrete user-review opportunities for the user comprises a historical information comparison opportunity.

Clause 8. The method of clause 7 wherein the historical plan comparison opportunity comprises a comparative presentation of dose information for at least one historical energy treatment session.

Clause 9. The method of clause 8 wherein the at least one historical energy treatment session dose information comprises at least one of: dose volume histogram estimates; three-dimensional dose predictions; clinical goal statistics.

Clause 10. The method of any of clause 1 through 9 wherein one of the plurality of discrete user-review opportunities for the user comprises an opportunity for the user to modify at least one automatically-generated energy treatment plan result.

Clause 11. An apparatus for generating an energy treatment plan for a particular patient using a particular energy treatment apparatus, the apparatus comprising: a control circuit that is operably coupled to a user interface, the control circuit configured to: access user-entered energy treatment plan-formation information; automatically generate an energy treatment plan for the particular patient using the particular energy treatment apparatus as a function of the user-entered energy treatment plan-formation information to provide an automatically-generated energy treatment plan; provide a plurality of discrete user-review opportunities for the user to evaluate the automatically-generated energy treatment plan, each of which plurality of discrete user-review opportunities does not include an opportunity for the user to select revisiting the user-entered energy treatment plan-formation information; subsequent to providing the plurality of discrete user-review opportunities, provide a discrete user opportunity that includes an opportunity for the user to select revisiting the user-entered energy treatment plan-formation information and thereby begin again development of the energy treatment plan.

Clause 12. The apparatus of clause 11 wherein the user-entered energy treatment plan-formation information includes at least one of patient target volume information, prescribed dose information, and dosing clinical goals.

Clause 13. The apparatus of either of clause 11 or 12 wherein the control circuit is configured to automatically generate the energy treatment plan for the particular patient using the particular energy treatment apparatus as a function of the user-entered energy treatment plan-formation information by optimization such that the automatically-generated energy treatment plan comprises an optimized energy treatment plan.

Clause 14. The apparatus of any of clause 11 through 13 wherein one of the plurality of discrete user-review opportunities for the user comprises an automatically-generated energy treatment plan quality review.

Clause 15. The apparatus of clause 14 wherein the automatically-generated energy treatment plan quality review comprises a presentation, via the user interface, of: dose-based patient imagery; clinical goals compliance; at least one dose-volume histogram.

Clause 16. The apparatus of clause 15 wherein the presentation comprises a read-only presentation that includes a user-opportunity to approve the automatically-generated energy treatment plan.

Clause 17. The apparatus of any of clause 11 through 16 wherein one of the plurality of discrete user-review opportunities for the user comprises a historical information comparison opportunity.

Clause 18. The apparatus of clause 17 wherein the historical plan comparison opportunity comprises a comparative presentation of dose information for at least one historical energy treatment session.

Clause 19. The apparatus of clause 18 wherein the at least one historical energy treatment session dose information comprises at least one of: dose volume histogram estimates; three-dimensional dose predictions; clinical goal statistics.

Clause 20. The apparatus of any of clause 11 through 19 wherein one of the plurality of discrete user-review opportunities for the user comprises an opportunity for the user to modify at least one automatically-generated energy treatment plan result.

Those skilled in the art will recognize that a wide variety of modifications, alterations, and combinations can be made with respect to the above described embodiments without departing from the scope of the invention. As one example in these regards, the straightforward simplicity of these teachings may work well with artificial intelligence techniques that may further enhance or guide a user's path through such a workflow. Accordingly, such modifications, alterations, and combinations are to be viewed as being within the ambit of the inventive concept.

1. A method for generating an energy treatment plan for a particular patient using a particular energy treatment apparatus, the method comprising:
by a control circuit that is operably coupled to a user interface:
accessing user-entered energy treatment plan-formation information;
automatically generating an energy treatment plan for the particular patient using the particular energy treatment apparatus as a function of the user-entered energy treatment plan-formation information to provide an automatically-generated energy treatment plan;
providing a plurality of discrete user-review opportunities for the user to evaluate the automatically-generated energy treatment plan, each of which plurality of discrete user-review opportunities does not include an opportunity for the user to select revisiting the user-entered energy treatment plan-formation information;
subsequent to providing the plurality of discrete user-review opportunities, providing a discrete user opportunity that includes an opportunity for the user to select revisiting the user-entered energy treatment plan-formation information and thereby begin again development of the energy treatment plan.

2. The method of claim 1 wherein the user-entered energy treatment plan-formation information includes at least one of patient target volume information, prescribed dose information, and dosing clinical goals.

3. The method of claim 1 wherein automatically generating the energy treatment plan for the particular patient using the particular energy treatment apparatus as a function of the user-entered energy treatment plan-formation information comprises optimization such that the automatically-generated energy treatment plan comprises an optimized energy treatment plan.

4. The method of claim 1 wherein one of the plurality of discrete user-review opportunities for the user comprises an automatically-generated energy treatment plan quality review.

5. The method of claim 4 wherein the automatically-generated energy treatment plan quality review comprises a presentation, via the user interface, of:
dose-based patient imagery;
clinical goals compliance;
at least one dose-volume histogram.

6. The method of claim 5 wherein the presentation comprises a read-only presentation that includes a user-opportunity to approve the automatically-generated energy treatment plan.

7. The method of claim 1 wherein one of the plurality of discrete user-review opportunities for the user comprises a historical information comparison opportunity.

8. The method of claim 7 wherein the historical plan comparison opportunity comprises a comparative presentation of dose information for at least one historical energy treatment session.

9. The method of claim 8 wherein the at least one historical energy treatment session dose information comprises at least one of:
dose volume histogram estimates;
three-dimensional dose predictions;
clinical goal statistics.

10. The method of claim 1 wherein one of the plurality of discrete user-review opportunities for the user comprises an opportunity for the user to modify at least one automatically-generated energy treatment plan result.

11. An apparatus for generating an energy treatment plan for a particular patient using a particular energy treatment apparatus, the apparatus comprising:
a control circuit that is operably coupled to a user interface, the control circuit configured to:
access user-entered energy treatment plan-formation information;
automatically generate an energy treatment plan for the particular patient using the particular energy treatment apparatus as a function of the user-entered energy treatment plan-formation information to provide an automatically-generated energy treatment plan;
provide a plurality of discrete user-review opportunities for the user to evaluate the automatically-generated energy treatment plan, each of which plurality of discrete user-review opportunities does not include an opportunity for the user to select revisiting the user-entered energy treatment plan-formation information;
subsequent to providing the plurality of discrete user-review opportunities, provide a discrete user opportunity that includes an opportunity for the user to select revisiting the user-entered energy treatment plan-formation information and thereby begin again development of the energy treatment plan.

12. The apparatus of claim 11 wherein the user-entered energy treatment plan-formation information includes at least one of patient target volume information, prescribed dose information, and dosing clinical goals.

13. The apparatus of claim 11 wherein the control circuit is configured to automatically generate the energy treatment plan for the particular patient using the particular energy treatment apparatus as a function of the user-entered energy treatment plan-formation information by optimization such that the automatically-generated energy treatment plan comprises an optimized energy treatment plan.

14. The apparatus of claim 11 wherein one of the plurality of discrete user-review opportunities for the user comprises an automatically-generated energy treatment plan quality review.

15. The apparatus of claim 14 wherein the automatically-generated energy treatment plan quality review comprises a presentation, via the user interface, of:
dose-based patient imagery;
clinical goals compliance;
at least one dose-volume histogram.

16. The apparatus of claim 15 wherein the presentation comprises a read-only presentation that includes a user-opportunity to approve the automatically-generated energy treatment plan.

17. The apparatus of claim 11 wherein one of the plurality of discrete user-review opportunities for the user comprises a historical information comparison opportunity.

18. The apparatus of claim 17 wherein the historical plan comparison opportunity comprises a comparative presentation of dose information for at least one historical energy treatment session.

19. The apparatus of claim 18 wherein the at least one historical energy treatment session dose information comprises at least one of:
dose volume histogram estimates;
three-dimensional dose predictions;
clinical goal statistics.

20. The apparatus of claim 11 wherein one of the plurality of discrete user-review opportunities for the user comprises an opportunity for the user to modify at least one automatically-generated energy treatment plan result.
